(19) United States
(12) Patent Application Publication
(10) Pub. No.: US 2026/0128133 A1
(43) Pub. Date: May 7, 2026

(54) DETERMINING INFORMATION ASSOCIATED WITH SYNTHESIZING A SOLID MIXTURE

(71) Applicant: Toyota Research Institute, Inc., Los Altos, CA (US)

(72) Inventor: Joseph Harold Montoya, Berkeley, CA (US)

(73) Assignees: Toyota Research Institute, Inc., Los Altos, CA (US); Toyota Jidosha Kabushiki Kaisha, Toyota-shi Aichi-ken (JP)

(21) Appl. No.: 18/940,176

(22) Filed: Nov. 7, 2024

(51) Int. Cl. G16C 20/10; G01N 23/2251

(52) U.S. Cl. CPC G16C 20/10; G01N 23/2251

(57) ABSTRACT

A system for determining information associated with synthesizing a solid mixture can include a processor and a memory. The memory can store a solid mixture sample assessment module, an electromagnetic beam source control module, and a radiation-impinged sample assessment module. The solid mixture sample assessment module can determine a set of positions on interfaces between grains in an initial sample of the solid mixture. The set can have maximal diversity of classifications of the interfaces in the initial sample. The electromagnetic beam source control module can cause electromagnetic beams to impinge the positions to produce an impinged sample. A characteristic of a first electromagnetic beam, which impinges a first position, can be different from the characteristic of a second electromagnetic beam, which impinges a second position. The radiation-impinged sample assessment module can analyze the positions on the impinged sample to determine the information associated with synthesizing the solid mixture.

### TECHNICAL FIELD

The disclosed technologies are directed to determining information associated with synthesizing a solid mixture.

### BACKGROUND

A chemical reaction can be a chemical transformation of one or more initial chemical compounds, referred to as reactants, into one or more other chemical compounds, referred to as products. Processes to execute, according to a sequence, one or more chemical reactions and, optionally, one or more physical manipulations to produce a specific end product can be referred to as synthesis. In synthesis, an initial or intermediate reactant can be referred to as a precursor. In some cases, when a specific end product is a solid, a physical manipulation performed on precursors of the specific end product can include production of a solid mixture. A solid mixture can be a material that includes two or more solids, which can be separated by one or more physical methods. Within a solid mixture, a continuous crystallite formation of one of the two or more solids can be referred to as a grain. A boundary between one grain and another grain can be referred to as an interface. Often, a specific end product can be produced by synthesizing a solid mixture. Because it can be possible that the specific end product can be produced by a variety of synthesis processes, it can be desirable to determine information associated with the variety of synthesis processes so that a specific synthesis process can be identified with respect to one or more of process efficiency, cost efficiency, time efficiency, or product yield.

### SUMMARY

In an embodiment, a system for determining information associated with synthesizing a solid mixture can include a processor and a memory. The memory can store a solid mixture sample assessment module, an electromagnetic beam source control module, and a radiation-impinged sample assessment module. The solid mixture sample assessment module can include instructions that, when executed by the processor, cause the processor to determine a set of positions on interfaces between grains in an initial sample of the solid mixture. The set can have maximal diversity of classifications of the interfaces in the initial sample. The electromagnetic beam source control module can include instructions that, when executed by the processor, cause electromagnetic beams to impinge the positions to produce an impinged sample. A characteristic of a first electromagnetic beam, which impinges a first position, can be different from the characteristic of a second electromagnetic beam, which impinges a second position. The radiation-impinged sample assessment module can include instructions that, when executed by the processor, cause the processor to analyze the positions on the impinged sample to determine the information associated with synthesizing the solid mixture.

In another embodiment, a method for determining information associated with synthesizing a solid mixture can include determining a set of positions on interfaces between grains in an initial sample of the solid mixture. The set can have maximal diversity of classifications of the interfaces in the initial sample. The method can include impinging, with electromagnetic beams, the positions to produce an impinged sample. A characteristic of a first electromagnetic beam, which impinges a first position, can be different from the characteristic of a second electromagnetic beam, which impinges a second position. The method can include analyzing the positions on the impinged sample to determine the information associated with synthesizing the solid mixture.

In another embodiment, a non-transitory computer-readable medium for determining information associated with synthesizing a solid mixture can include instructions that, when executed by one or more processors, cause the one or more processors to determine a set of positions on interfaces between grains in an initial sample of the solid mixture. The set can have maximal diversity of classifications of the interfaces in the initial sample. The non-transitory computer-readable medium can include instructions that, when executed by one or more processors, cause the one or more processors to cause electromagnetic beams to impinge the positions to produce an impinged sample. A characteristic of a first electromagnetic beam, which impinges a first position, can be different from the characteristic of a second electromagnetic beam, which impinges a second position. The non-transitory computer-readable medium can include instructions that, when executed by one or more processors, cause the one or more processors to analyze the positions on the impinged sample to determine the information associated with synthesizing the solid mixture.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, groups of boxes, or other shapes) in the figures represent one embodiment of the boundaries. In some embodiments, one element may be designed as multiple elements or multiple elements may be designed as one element. In some embodiments, an element shown as an internal component of another element may be implemented as an external component and vice versa. Furthermore, elements may not be drawn to scale.

FIG. 1 includes a block diagram that illustrates an example of an environment for determining information associated with synthesizing a solid mixture, according to the disclosed technologies.

FIG. 2 includes a block diagram that illustrates an example of a system for determining information associated with synthesizing a solid mixture, according to the disclosed technologies.

FIG. 3 includes a diagram that illustrates an example of an image of an initial sample of the solid mixture.

FIG. 4 includes a diagram that illustrates an example of an image that includes the image illustrated in FIG. 3 and labels for classifications of interfaces and grains.

FIG. 5 includes a diagram that illustrates an example of an image that includes the image illustrated in FIG. 3 and a set of positions on the interfaces to be impinged by electromagnetic beams, according to the disclosed technologies.

FIG. 6 includes a diagram that illustrates an example of an image of an impinged sample of the solid mixture.

FIG. 7 includes a diagram that illustrates an example of a table of a database, according to the disclosed technologies.

FIGS. 8A and 8B include a flow diagram that illustrates an example of a method that is associated with determining information associated with synthesizing a solid mixture, according to the disclosed technologies.

### DETAILED DESCRIPTION

The disclosed technologies are directed to determining information associated with synthesizing a solid mixture. A set of positions on interfaces between grains in an initial sample of the solid mixture can be determined. The set can have maximal diversity of classifications of the interfaces in the initial sample. For example, an image of the initial sample can be produced, and the set of the positions can be determined by analyzing the image of the initial sample. For example, the image of the initial sample can be produced using a scanning transmission electron microscopy technique. For example, the image of the initial sample can be analyzed by processing, using an artificial intelligence technique, the image of the initial sample. For example, a classification of an interface can include a first classification and a second classification. For example: (1) the first classification can be for a family of lattice planes of a first material at the interface and (2) the second classification can be for a family of lattice planes of a second material at the interface.

The positions can be impinged with electromagnetic beams to produce an impinged sample. For example, a calibration of the image of the initial sample can be retained and, using the calibration, the positions can be impinged with the electromagnetic beams. A characteristic of a first electromagnetic beam, which impinges a first position, can be different from the characteristic of a second electromagnetic beam, which impinges a second position. For example, the characteristic can include one or more of: (1) a bias voltage applied to a source of an electromagnetic beam or (2) a size of a cross section of the electromagnetic beam. For example, the set of the positions on the interfaces can also have maximal diversity of the characteristics of the electromagnetic beams that impinge the positions. For example, a source of one or more of the electromagnetic beams can include a scanning transmission electron microscope. For example, the scanning transmission electron microscope can be the scanning transmission electron microscope used to produce the image of the initial sample. For example: (1) one or more first bias voltages can be applied to the scanning transmission electron microscope to produce the image of the initial sample and (2) one or more second bias voltages can be applied to the scanning transmission electron microscope to impinge the positions.

For example, a bias voltage applied to a source of the electromagnetic beam to impinge a position can be based on thermodynamic data associated with synthesizing a solid mixture. For example, based on the bias voltage applied to the source of the electromagnetic beam that impinged the position: (1) a temperature of a heat treatment associated with a production, using a synthesis technique, of the solid mixture and (2) a duration of time of the heat treatment can be determined. By having a set of positions on interfaces between grains in a sample of a solid mixture impinged with electromagnetic beams in a manner in which there can be: (1) maximal diversity of classifications of the interfaces in the sample of the solid mixture and (2) maximal diversity of characteristics of the electromagnetic beams, a large amount of information associated with synthesizing the solid mixture can be determined from a single sample of the solid mixture, which can avoid a need to produce several samples in order to determine such a large amount of information.

The positions on the impinged sample can be analyzed to determine the information associated with synthesizing the solid mixture. For example, an image of the impinged sample can be produced and the positions on the impinged sample, in the image of the impinged sample, can be analyzed to determine the information associated with synthesizing the solid mixture. For example, the image of the impinged sample can be produced using a scanning transmission electron microscopy technique. For example, the scanning transmission electron microscope can be the scanning transmission electron microscope used to impinge the positions. For example, one or more first bias voltages can be applied to the scanning transmission electron microscope to impinge the positions, and one or more second bias voltages can be applied to the scanning transmission electron microscope to produce the image of the impinged sample. For example, the image of the impinged sample can be analyzed by processing, using an artificial intelligence technique, the image of the impinged sample. For example, the information associated with synthesizing the solid mixture can include one or more of a result of a determination that the position is one of crystalline or amorphous, a result composition, a matching crystalline phase, an X-ray diffraction pattern, or the like.

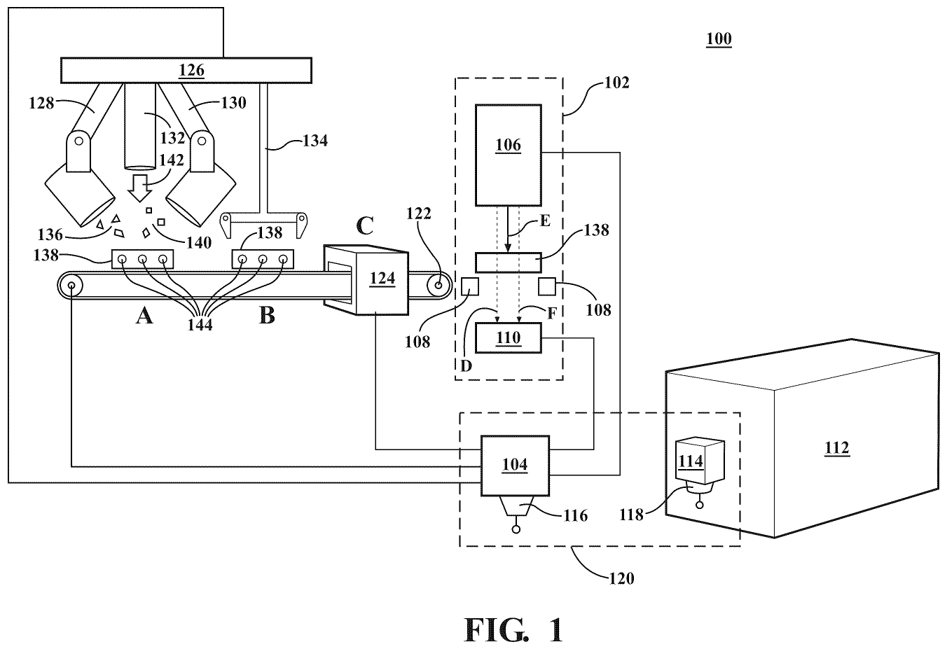

FIG. 1 includes a block diagram that illustrates an example of an environment 100 for determining information associated with synthesizing a solid mixture, according to the disclosed technologies. The environment 100 can include, for example, an electromagnetic beam management system 102 and a first controller 104. For example, the electromagnetic beam management system 102 can include an electromagnetic beam management source 106, a stage 108, and a detector 110. For example, the first controller 104 can be communicably coupled to the electromagnetic beam management source 106. For example, the first controller 104 can be communicably coupled to the detector 110.

Additionally, for example, the environment 100 can include synthesis apparatus 112 and a second controller 114. For example, the second controller 114 can be communicably coupled to the synthesis apparatus 112. For example: (1) the first controller 104 can include a communications device 116 and (2) the second controller 114 can include a communications device 118. For example, data can be communicated, via the communications device 116 and the communications device 118, between the first controller 104 and the second controller 114. Alternatively, for example, a combined controller 120 can include the first controller 104 and the second controller 114.

Additionally, for example, the environment 100 can include a conveyor system 122 and a furnace 124. For example, the first controller 104 can be communicably coupled to the conveyor system 122. For example, the first controller 104 can be communicably coupled to the furnace 124.

Additionally, for example, the environment 100 can include a robotic system 126. For example, the first controller 104 can be communicably coupled to the robotic system 126. For example, the robotic system 126 can include a first robotic arm 128, a second robotic arm 130, a dispenser 132, and a rotator 134.

For example, a sequence to determine information associated with synthesizing a solid mixture can include the following phases. In a Phase A, for example: (1) the first robotic arm 128 can cause a first material 136 to be put into a container 138, (2) the second robotic arm 130 can cause a second material 140 to be put into the container 138, and (3) the dispenser 132 can cause a solvent 142 to be put into the container 138. For example, the container 138 can also contain balls 144 to be used to mix, using a ball milling technique, the first material 136 and the second material 140. In a Phase B, for example, the rotator 134 can cause the container 138 to rotate the container 138 to mix the first material 136 and the second material 140. In a Phase C, for example: (1) the container 138 can be put in the furnace 124 and (2) the furnace 124 can heat the first material 136 and the second material 140 to produce the solid mixture. In a Phase D, for example: (1) the container 138, which contains an initial sample of the solid mixture, can be put on the stage 108, (2) the electromagnetic beam management source 106 can cause an image of the initial sample to be produced on the detector 110, and (3) the image of the initial sample can be analyzed to determine positions on interfaces between grains in the initial sample. In a Phase E, for example, the electromagnetic beam management source 106 can cause electromagnetic beams to impinge the positions to produce an impinged sample. In a Phase F, for example: (1) the electromagnetic beam management source 106 can cause an image of the impinged sample to be produced on the detector 110 and (2) the image of the impinged sample can be analyzed to determine the information associated with synthesizing the solid mixture.

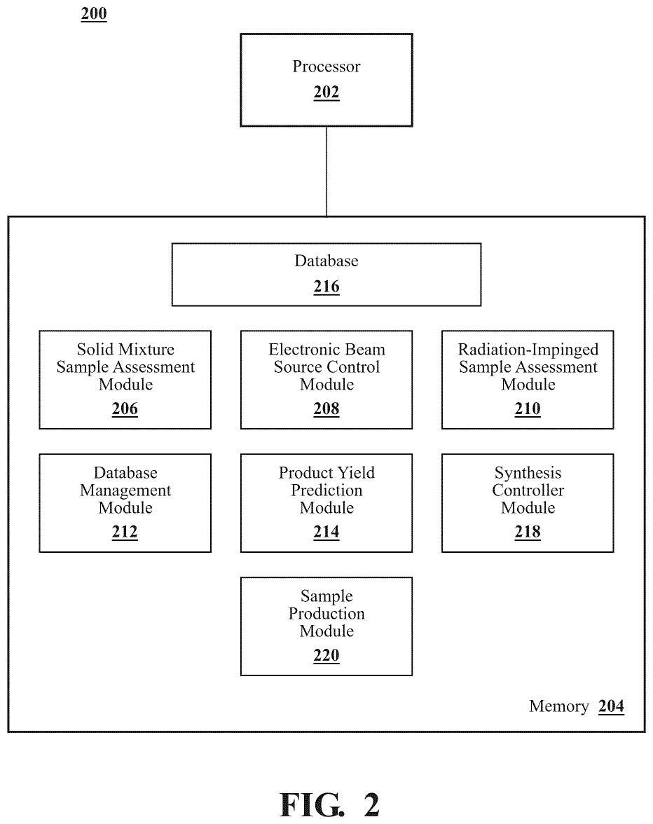

FIG. 2 includes a block diagram that illustrates an example of a system 200 for determining information associated with synthesizing a solid mixture, according to the disclosed technologies. The system 200 can include, for example, a processor 202 and a memory 204. The memory 204 can be communicably coupled to the processor 202. For example, the memory 204 can store a solid mixture sample assessment module 206, an electromagnetic beam source control module 208, and a radiation-impinged sample assessment module 210. For example, the first controller 104 (illustrated in FIG. 1) can be configured to include the system 200.

For example, the solid mixture sample assessment module 206 can include instructions that function to control the processor 202 to determine a set of positions on interfaces between grains in an initial sample of the solid mixture. The set can have maximal diversity of classifications of the interfaces in the sample.

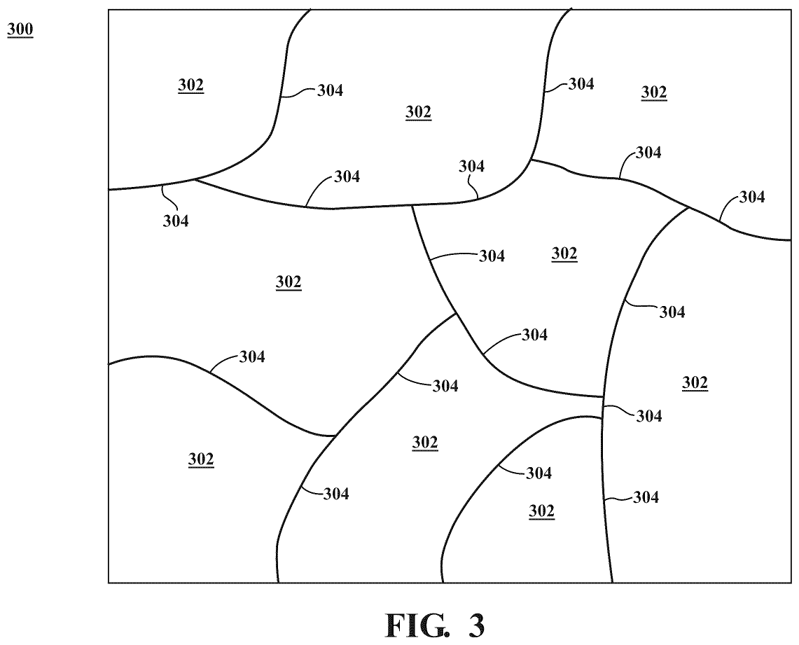

For example, the solid mixture sample assessment module 206 can further include instructions to produce an image of the initial sample. FIG. 3 includes a diagram that illustrates an example of an image 300 of an initial sample of the solid mixture. For example, the image 300 can include: (1) grains 302 of two or more solid state materials and (2) interfaces 304 between the grains 302. For example, the instructions to produce the image 300 of the initial sample can include instructions to produce, using a ptychographic technique, the image 300 of the initial sample. Alternatively or additionally, for example, the instructions to produce the image 300 of the initial sample can include instructions to produce, using an energy-dispersive X-ray spectroscopy technique, the image 300 of the initial sample. Alternatively or additionally, for example, the instructions to produce the image 300 of the initial sample can include instructions to produce, using a scanning transmission electron microscopy technique, the image 300 of the initial sample. For example, the scanning transmission electron microscopy technique can include a 4D scanning transmission electron microscopy technique.

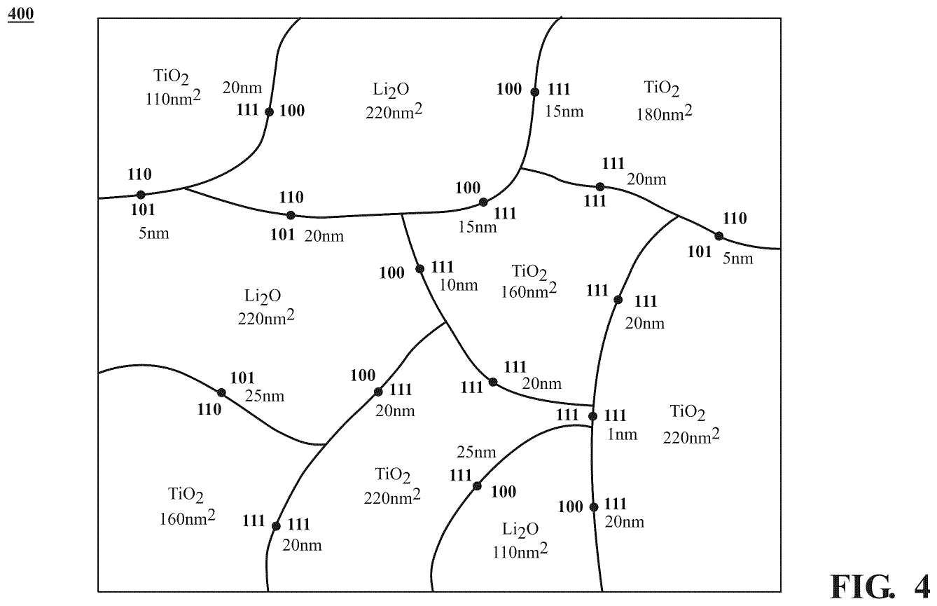

For example, the instructions to determine the set of the positions can include instructions to analyze the image 300 of the initial sample to determine the set of the positions. For example, the instructions to analyze the image 300 of the initial sample can include instructions to process, using an artificial intelligence technique, the image 300 of the initial sample. For example, the artificial intelligence technique can include a computer vision technique. For example, the artificial intelligence technique can use a neural network. For example, the artificial intelligence technique can segment the image 300 into objects. For example, the solid mixture sample assessment module can further include instructions to classify the objects in the image 300 of the initial sample. For example, the objects can include the interfaces. For example, the instructions to classify the objects can include instructions to process, using an artificial intelligence technique, the image 300 of the initial sample. For example, the artificial intelligence technique can include a computer vision technique. For example, the artificial intelligence technique can use a neural network. For example, a classification of an interface can include a first classification and a second classification. For example: (1) the first classification can be for a family of lattice planes of a first material at the interface and (2) the second classification can be for a family of lattice planes of a second material at the interface. For example, the family of lattice planes can be identified by Miller indices. For example, the classification of the interface can further include a value of a length of the interface. For example, the objects can further include grains of materials. For example, a classification of a grain of a material can include: (1) a chemical formula of the material and (2) a value of an area of the grain of the material. For example, the artificial intelligence technique can label the classifications of the interfaces, the grains of material, or both. FIG. 4 includes a diagram that illustrates an example of an image 400 that includes the image 300 and labels for the classifications of the interfaces 304 and the grains 302.

Returning to FIG. 2, for example, the electromagnetic beam source control module 208 can include instructions that function to control the processor 202 to cause electromagnetic beams to impinge the positions to produce an impinged sample. A characteristic of a first electromagnetic beam, which impinges a first position, can be different from the characteristic of a second electromagnetic beam, which impinges a second position. For example, the characteristic can include one or more of: (1) a bias voltage applied to a source of an electromagnetic beam or (2) a size of a cross section of the electromagnetic beam. For example, the bias voltage can be based on thermodynamic data associated with synthesizing the solid mixture. For example, the set of the positions on the interfaces can have maximal diversity of the characteristics of the electromagnetic beams that impinge the positions. For example: (1) the solid mixture sample assessment module 206 can further include instructions to retain a calibration of the image of the initial sample and (2) the instructions to cause the electromagnetic beams to impinge the positions can include instructions to cause, using the calibration, the electromagnetic beams to impinge the positions.

For example, one or more of the electromagnetic beams can include one or more of an electron beam, an ion beam, or an X-ray beam. For example, the instructions to cause the electromagnetic beams to impinge the positions can include instructions to cause a source of an electromagnetic beam to move in a raster pattern to cause the electromagnetic beams to impinge the positions. Alternatively or additionally, for example, the instructions to cause the electromagnetic beams to impinge the positions can include instructions to cause multiple electromagnetic beams to impinge, concurrently, multiple positions of the positions. Alternatively or additionally, for example, the instructions to cause the electromagnetic beams to impinge the positions can include instructions to cause a position to be impinged with two or more of the electron beam, the ion beam, or the X-ray beam. For example, a source of one or more of the electromagnetic beams can include a scanning transmission electron microscope. For example, the scanning transmission electron microscope can be the scanning transmission electron microscope used to produce the image of the initial sample. For example: (1) one or more first bias voltages can be applied to the scanning transmission electron microscope to produce the image of the initial sample and (2) one or more second bias voltages can be applied to the scanning transmission electron microscope to impinge the positions. For example, the system 200 can be configured so that the initial sample remains at a same location on a stage (e.g., the stage 108 illustrated in FIG. 1) of the scanning transmission electron microscope (e.g., the electromagnetic beam management source 106 illustrated in FIG. 1) during a production of the image of the initial sample and during an impingement of the positions.

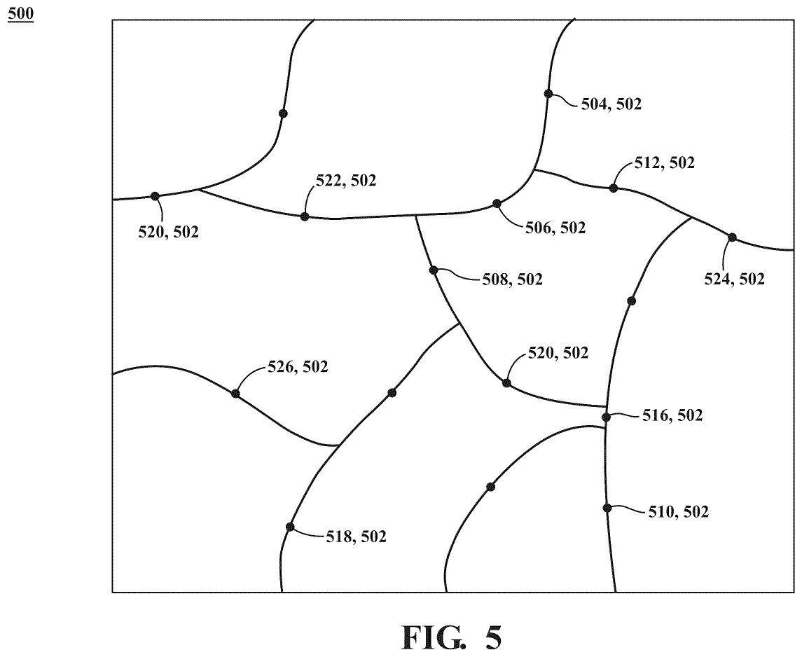

FIG. 5 includes a diagram that illustrates an example of an image 500 that includes the image 300 and the set 502 of the positions on the interfaces to be impinged by the electromagnetic beams, according to the disclosed technologies. For example, the set 502 of the positions on the interfaces can have maximal diversity of: (1) the classifications of the interfaces and (2) the characteristics of the electromagnetic beams. For example, the characteristics of the electromagnetic beams can include: (1) a bias voltage applied to a source of an electromagnetic beam or (2) a size of a cross section of the electromagnetic beam. For example, the set 502 of the positions on the interfaces can include: (1) a first position 504 having an interface classified as 100-111, a bias voltage of 1.2V, and a size of a cross section of 0.05 nm, (2) a second position 506 having an interface classified as 100-111, a bias voltage of 1.2V, and a size of a cross section of 0.20 nm, (3) a third position 508 having an interface classified as 100-111, a bias voltage of 5.0V, and a size of a cross section of 0.05 nm, (4) a fourth position 510 having an interface classified as 100-111, a bias voltage of 5.0V, and a size of a cross section of 0.20 nm, (5) a fifth position 512 having an interface classified as 111-111, a bias voltage of 1.2V, and a size of a cross section of 0.05 nm, (6) a sixth position 514 having an interface classified as 111-111, a bias voltage of 1.2V, and a size of a cross section of 0.20 nm, (7) a seventh position 516 having an interface classified as 111-111, a bias voltage of 5.0V, and a size of a cross section of 0.05 nm, (8) an eighth position 518 having an interface classified as 111-111, a bias voltage of 5.0V, and a size of a cross section of 0.20 nm, (9) a ninth position 520 having an interface classified as 101-110, a bias voltage of 1.2V, and a size of a cross section of 0.05 nm, (10) a tenth position 522 having an interface classified as 101-110, a bias voltage of 1.2V, and a size of a cross section of 0.20 nm, (11) an eleventh position 524 having an interface classified as 101-110, a bias voltage of 5.0V, and a size of a cross section of 0.05 nm, and (12) a twelfth position 526 having an interface classified as 101-110, a bias voltage of 5.0V, and a size of a cross section of 0.20 nm.

Returning to FIG. 2, for example, the radiation-impinged sample assessment module 210 can include instructions that function to control the processor 202 to analyze the positions on the impinged sample to determine the information associated with synthesizing the solid mixture. For example: (1) the radiation-impinged sample assessment module 210 can further include instructions to produce an image of the impinged sample and (2) the instructions to analyze the positions on the impinged sample to determine the information associated with synthesizing the solid mixture can include instructions to analyze the positions on the impinged sample, in the image of the impinged sample, to determine the information associated with synthesizing the solid mixture. For example, the information associated with synthesizing the solid mixture can include one or more of a result of a determination that the position is one of crystalline or amorphous, a result composition, a matching crystalline phase, an X-ray diffraction pattern, or the like.

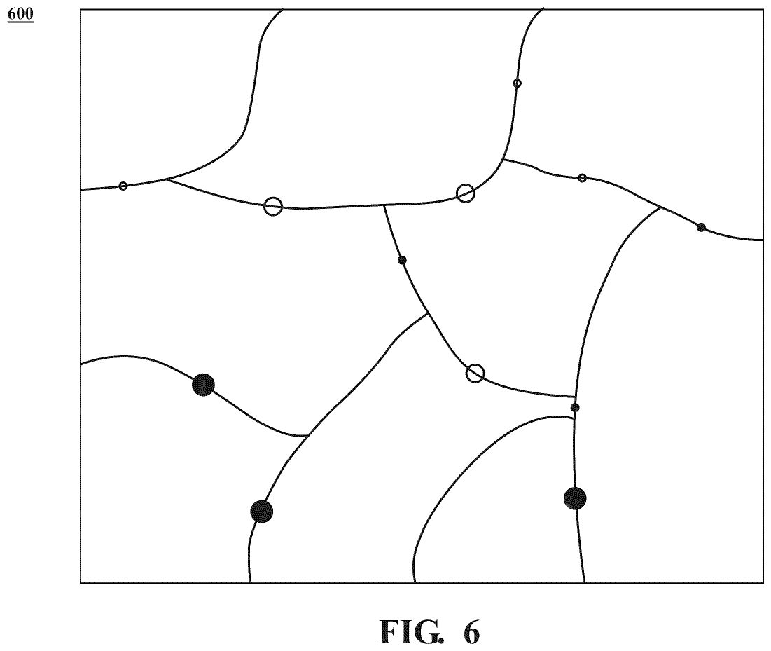

FIG. 6 includes a diagram that illustrates an example of an image 600 of an impinged sample of the solid mixture. For example, the instructions to produce the image 600 of the impinged sample can include instructions to produce, using a ptychographic technique, the image 600 of the impinged sample. Alternatively or additionally, for example, the instructions to produce the image 600 of the impinged sample can include instructions to produce, using an energy-dispersive X-ray spectroscopy technique, the image 600 of the impinged sample. Alternatively or additionally, for example, the instructions to produce the image 600 of the impinged sample can include instructions to produce, using a scanning transmission electron microscopy technique, the image 600 of the impinged sample. For example, the scanning transmission electron microscopy technique can include a 4D scanning transmission electron microscopy technique.

For example, a source of one or more of the electromagnetic beams can include a scanning transmission electron microscope. For example, the scanning transmission electron microscope can be the scanning transmission electron microscope used to impinge the positions. For example: (1) one or more first bias voltages can be applied to the scanning transmission electron microscope to impinge the positions and (2) one or more second bias voltages can be applied to the scanning transmission electron microscope to produce the image of the impinged sample. For example, the system 200 can be configured so that the initial sample remains at a same location on a stage (e.g., the stage 108 illustrated in FIG. 1) of the scanning transmission electron microscope (e.g., the electromagnetic beam management source 106 illustrated in FIG. 1) during an impingement of the positions and during a production of the image of the impinged sample.

For example, the instructions to analyze the positions on the impinged sample in the image 600 of the impinged sample include instructions to process, using an artificial intelligence technique, the image 600 of the impinged sample. For example, the artificial intelligence technique can include a computer vision technique. For example, the artificial intelligence technique can use a neural network.

Returning to FIG. 2, for example, the memory 204 can further store a database management module 212 and a product yield prediction module 214. The database management module 212 can include instructions that function to control the processor 202 to store, in a database 216 and for a position, first data. For example, the first data can include: (1) the information associated with synthesizing the solid mixture, (2) a classification of an interface, and (3) the characteristic of an electromagnetic beam that impinged the position. For example, the information associated with synthesizing the solid mixture can include one or more of a result of a determination that the position is one of crystalline or amorphous, a result composition, a matching crystalline phase, an X-ray diffraction pattern, or the like.

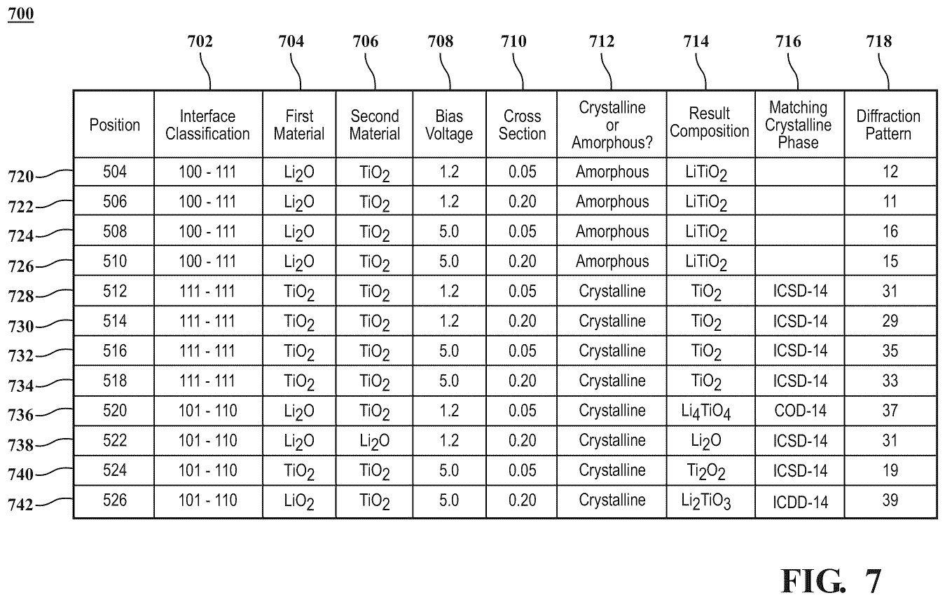

FIG. 7 includes a diagram that illustrates an example of a table 700 of a database, according to the disclosed technologies. For example, the database can be the database 216 illustrated in FIG. 2. For example, the table 700 can include fields for the classification of the interface 702, an identification of the first material 704, an identification of the second material 706, the bias voltage applied to the source of the electromagnetic beam 708, the size of the cross section of the electromagnetic beam 710, the result of the determination that the position is one of crystalline or amorphous 712, the result composition 714, the matching crystalline phase 716, and the X-ray diffraction pattern 718. With reference to FIGS. 4, 5, and 7, for example, the table 700 can include a first record 720 for the first position 504, a second record 722 for the second position 506, a third record 724 for the third position 508, a fourth record 726 for the fourth position 510, a fifth record 728 for the fifth position 512, a sixth record 730 for the sixth position 514, a seventh record 732 for the seventh position 516, an eighth record 734 for the eighth position 518, a ninth record 736 for the ninth position 520, a tenth record 738 for the tenth position 522, an eleventh record 740 for the eleventh position 524, and a twelfth record 742 for the twelfth position 526.

Returning to FIG. 2, for example, the product yield prediction module 214 can include instructions that function to control the processor 202 to: (1) obtain second data and (2) determine, using the first data and the second data, a prediction of a product yield of the production, using a synthesis technique, of the solid mixture. For example, the second data can be associated with a production, using the synthesis technique, of the solid mixture. For example, the second data can include one or more of: (1) an identity of a solvent used to process two or more solid state materials to produce the initial sample of the solid mixture, (2) a speed of a ball milling device used to produce the initial sample of the solid mixture, or (3) a profile of a heat treatment associated with the production, using the synthesis technique, of the solid mixture. For example, the profile can include a temperature of the heat treatment and a duration of time of the heat treatment. For example, the synthesis technique can be one or more processes associated with producing the solid mixture at a mass or a volume associated with industrial applications. For example, the product yield prediction module can further include instructions to determine, based on a bias voltage applied to a source of the electromagnetic beam that impinged the position, the temperature of the heat treatment and the duration of time of the heat treatment. For example, the instructions to determine the prediction of the product yield can include instructions to determine, using a machine learning technique, the prediction of the product yield. For example, the machine learning technique can include an unsupervised machine learning technique. For example, the machine learning technique can use a neural network.

For example, the memory 204 can further store a synthesis controller module 218. The synthesis controller module 218 can include instructions that function to control the processor 202 to control, using the first data and the second data, the synthesis technique to produce the solid mixture. For example, the synthesis apparatus 112 (illustrated in FIG. 1) can be configured to perform the synthesis technique. For example, the combined controller 120 (illustrated in FIG. 1) can be configured to include the system 200.

Alternatively, for example, the synthesis controller module 218 can further includes instruction to communicate the first data and the second data to a controller configured to control the synthesis technique to produce the solid mixture. For example, the first controller 104 (illustrated in FIG. 1) can be configured to include the system 200. For example, the second controller 114 (illustrated in FIG. 1) can be the controller configured to control the synthesis technique to produce the solid mixture. For example, the first controller 104, via the communications device 116 (illustrated in FIG. 1), can communicate the first data and the second data to the second controller 114 via the communications device 118 (illustrated in FIG. 1).

For example, the memory 204 can further store a sample production module 220. The sample production module 220 can include instructions that function to control the processor 202 to cause the system 200 to produce the initial sample of the solid mixture.

For example, the instructions to cause the system 200 to produce the initial sample of the solid mixture can include instructions to cause the system 200 to: (1) process, using a solvent, two or more solid state materials, (2) mix the two or more solid state materials, and (3) heat the two or more solid state materials. For example, the solvent can include ethanol (C₂H₆O). For example, the two or more solid state materials can include titanium dioxide (TiO₂) and lithium oxide (Li₂O). For example, the instructions to cause the system 200 to mix the two or more solid state materials can include instructions to cause the system to mix, using a ball milling technique, the two or more solid state materials. For example, a specific end product produced by synthesizing the solid mixture can include lithium titanate (Li₂TiO₃).

Alternatively or additionally, for example, the instructions to cause the system 200 to produce the initial sample of the solid mixture can include instructions to cause the system 200 to produce, using a robotic technique, the initial sample of the solid mixture. For example, the robotic technique can include moving the initial sample of the solid mixture to a stage of an electromagnetic beam management system. For example, the system 200 can be configured to include the conveyor system 122 (illustrated in FIG. 1), the furnace 124 (illustrated in FIG. 1), and the robotic system 126 (illustrated in FIG. 1).

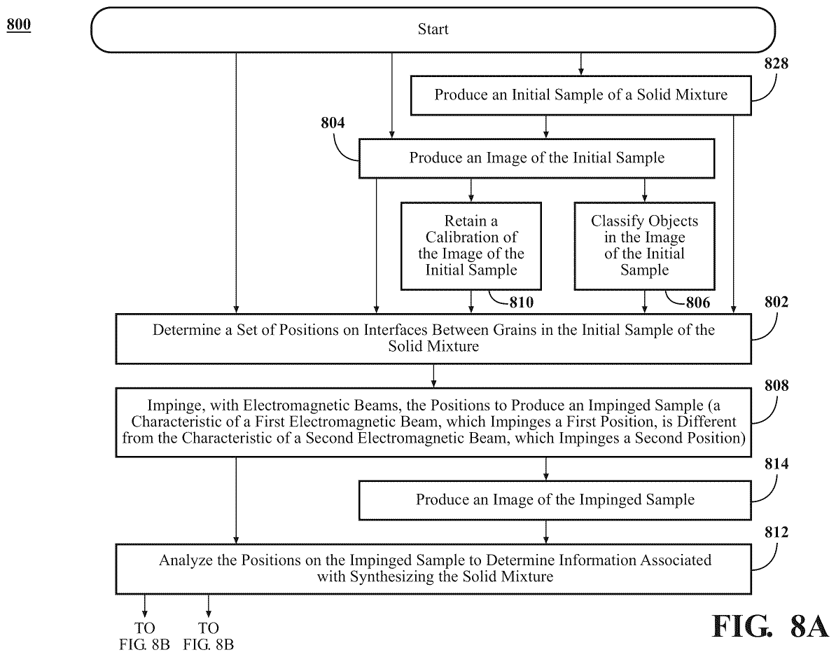

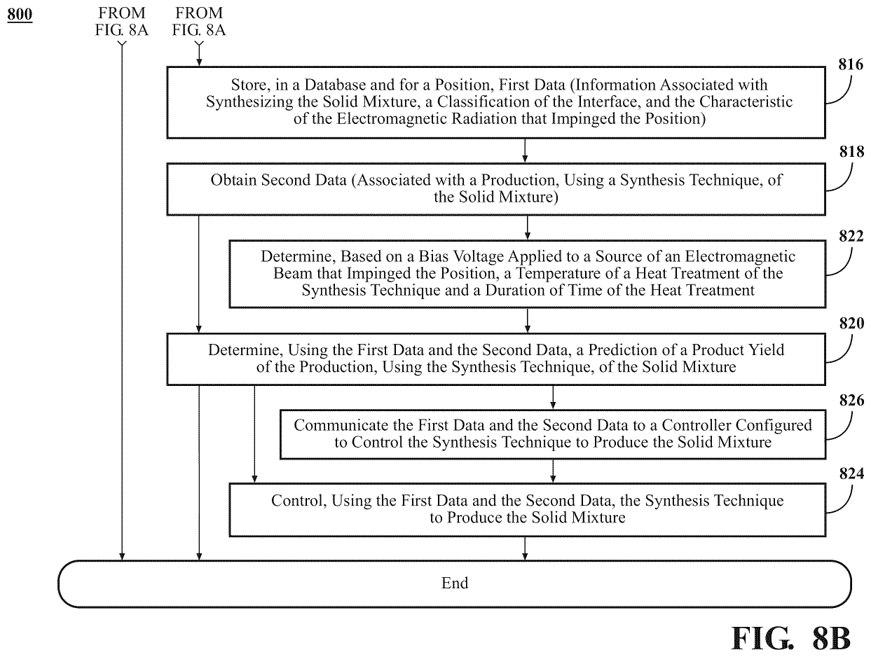

FIGS. 8A and 8B include a flow diagram that illustrates an example of a method 800 that is associated with determining information associated with synthesizing a solid mixture, according to the disclosed technologies. Although the method 800 is described in combination with the system 200 illustrated in FIG. 2, one of skill in the art understands, in light of the description herein, that the method 800 is not limited to being implemented by the system 200 illustrated in FIG. 2. Rather, the system 200 illustrated in FIG. 2 is an example of a system that may be used to implement the method 800. Additionally, although the method 800 is illustrated as a generally serial process, various aspects of the method 800 may be able to be executed in parallel.

In FIG. 8A, in the method 800, at an operation 802, for example, the solid mixture sample assessment module 206 can determine a set of positions on interfaces between grains in an initial sample of the solid mixture. The set can have maximal diversity of classifications of the interfaces in the sample.

Additionally, at an operation 804 for example, the solid mixture sample assessment module 206 can produce an image of the initial sample. For example, at the operation 804, the solid mixture sample assessment module 206 can produce, using a ptychographic technique, the image of the initial sample. Alternatively or additionally, for example, at the operation 804, the solid mixture sample assessment module 206 can produce, using an energy-dispersive X-ray spectroscopy technique, the image of the initial sample. Alternatively or additionally, for example, at the operation 804, the solid mixture sample assessment module 206 can produce, using a scanning transmission electron microscopy technique, the image of the initial sample. For example, the scanning transmission electron microscopy technique can include a 4D scanning transmission electron microscopy technique.

For example, at the operation 802, the solid mixture sample assessment module 206 can analyze the image of the initial sample to determine the set of the positions. For example, at the operation 802, the solid mixture sample assessment module 206 can process, using an artificial intelligence technique, the image of the initial sample. For example, the artificial intelligence technique can include a computer vision technique. For example, the artificial intelligence technique can use a neural network. For example, the artificial intelligence technique can segment the image into objects.

Additionally, at an operation 806 for example, the solid mixture sample assessment module 206 can classify the objects in the image of the initial sample. For example, the objects can include the interfaces. For example, at the operation 806, the solid mixture sample assessment module 206 can process, using an artificial intelligence technique, the image of the initial sample. For example, the artificial intelligence technique can include a computer vision technique. For example, the artificial intelligence technique can use a neural network. For example, a classification of an interface can include a first classification and a second classification. For example: (1) the first classification can be for a family of lattice planes of a first material at the interface and (2) the second classification can be for a family of lattice planes of a second material at the interface. For example, the family of lattice planes can be identified by Miller indices. For example, the classification of the interface can further include a value of a length of the interface. For example, the objects can further include grains of materials. For example, a classification of a grain of a material can include: (1) a chemical formula of the material and (2) a value of an area of the grain of the material. For example, the artificial intelligence technique can label the classifications of the interfaces, the grains of material, or both.

At an operation 808, for example, the electromagnetic beam source control module 208 can cause electromagnetic beams to impinge the positions to produce an impinged sample. A characteristic of a first electromagnetic beam, which impinges a first position, can be different from the characteristic of a second electromagnetic beam, which impinges a second position. For example, the characteristic can include one or more of: (1) a bias voltage applied to a source of an electromagnetic beam or (2) a size of a cross section of the electromagnetic beam. For example, the bias voltage can be based on thermodynamic data associated with synthesizing the solid mixture. For example, the set of the positions on the interfaces can have maximal diversity of the characteristics of the electromagnetic beams that impinge the positions.

Additionally, at an operation 810 for example, the solid mixture sample assessment module 206 can retain a calibration of the image of the initial sample.

For example, at the operation 808, the electromagnetic beam source control module 208 can cause, using the calibration, the electromagnetic beams to impinge the positions.

For example, one or more of the electromagnetic beams can include one or more of an electron beam, an ion beam, or an X-ray beam. For example, at the operation 808, the electromagnetic beam source control module 208 can cause a source of an electromagnetic beam to move in a raster pattern to cause the electromagnetic beams to impinge the positions. Alternatively or additionally, for example, at the operation 808, the electromagnetic beam source control module 208 can cause multiple electromagnetic beams to impinge, concurrently, multiple positions of the positions. Alternatively or additionally, for example, at the operation 808, the electromagnetic beam source control module 208 can cause a position to be impinged with two or more of the electron beam, the ion beam, or the X-ray beam. For example, a source of one or more of the electromagnetic beams can include a scanning transmission electron microscope. For example, the scanning transmission electron microscope can be the scanning transmission electron microscope used, at the operation 804, to produce the image of the initial sample. For example: (1) one or more first bias voltages can be applied to the scanning transmission electron microscope to produce the image of the initial sample and (2) one or more second bias voltages can be applied to the scanning transmission electron microscope to impinge the positions. For example, the system 200 can be configured so that the initial sample remains at a same location on a stage (e.g., the stage 108 illustrated in FIG. 1) of the scanning transmission electron microscope (e.g., the electromagnetic beam management source 106 illustrated in FIG. 1) during a production of the image of the initial sample (at the operation 804) and during an impingement of the positions (at the operation 808).

At an operation 812, for example, the radiation-impinged sample assessment module 210 can analyze the positions on the impinged sample to determine the information associated with synthesizing the solid mixture.

Additionally, at an operation 814, for example, the radiation-impinged sample assessment module 210 can produce an image of the impinged sample. For example, at the operation 812, the radiation-impinged sample assessment module 210 can analyze the positions on the impinged sample, in the image of the impinged sample, to determine the information associated with synthesizing the solid mixture. For example, the information associated with synthesizing the solid mixture can include one or more of a result of a determination that the position is one of crystalline or amorphous, a result composition, a matching crystalline phase, an X-ray diffraction pattern, or the like.

For example, at the operation 814, the radiation-impinged sample assessment module 210 can produce, using a ptychographic technique, the image of the impinged sample. Alternatively or additionally, for example, at the operation 814, the radiation-impinged sample assessment module 210 can produce, using an energy-dispersive X-ray spectroscopy technique, the image of the impinged sample. Alternatively or additionally, for example, at the operation 814, the radiation-impinged sample assessment module 210 can produce, using a scanning transmission electron microscopy technique, the image of the impinged sample. For example, the scanning transmission electron microscopy technique can include a 4D scanning transmission electron microscopy technique.

For example, a source of one or more of the electromagnetic beams can include a scanning transmission electron microscope. For example, the scanning transmission electron microscope can be the scanning transmission electron microscope used, at the operation 808, to impinge the positions. For example: (1) one or more first bias voltages can be applied to the scanning transmission electron microscope to impinge the positions and (2) one or more second bias voltages can be applied to the scanning transmission electron microscope to produce the image of the impinged sample. For example, the system 200 can be configured so that the initial sample remains at a same location on a stage (e.g., the stage 108 illustrated in FIG. 1) of the scanning transmission electron microscope (e.g., the electromagnetic beam management source 106 illustrated in FIG. 1) during an impingement of the positions (at the operation 808) and during a production of the image of the impinged sample (at the operation 814).

For example, at the operation 812, the radiation-impinged sample assessment module 210 can process, using an artificial intelligence technique, the image of the impinged sample. For example, the artificial intelligence technique can include a computer vision technique. For example, the artificial intelligence technique can use a neural network.

Additionally, in the FIG. 8B, in the method 800, at an operation 816, for example, the database management module 212 can store, in a database 216 and for a position, first data. For example, the first data can include: (1) the information associated with synthesizing the solid mixture, (2) a classification of an interface, and (3) the characteristic of an electromagnetic beam that impinged the position. For example, the information associated with synthesizing the solid mixture can include one or more of a result of a determination that the position is one of crystalline or amorphous, a result composition, a matching crystalline phase, an X-ray diffraction pattern, or the like.

Additionally, at an operation 818, for example, the product yield prediction module 214 can obtain second data. For example, the second data can be associated with a production, using the synthesis technique, of the solid mixture. For example, the second data can include one or more of: (1) an identity of a solvent used to process two or more solid state materials to produce the initial sample of the solid mixture, (2) a speed of a ball milling device used to produce the initial sample of the solid mixture, or (3) a profile of a heat treatment associated with the production, using the synthesis technique, of the solid mixture. For example, the profile can include a temperature of the heat treatment and a duration of time of the heat treatment. For example, the synthesis technique can be one or more processes associated with producing the solid mixture at a mass or a volume associated with industrial applications.

Additionally, at an operation 820, for example, the product yield prediction module 214 can determine, using the first data and the second data, a prediction of a product yield of the production, using a synthesis technique, of the solid mixture.

Additionally, at an operation 822, for example, the product yield prediction module 214 can determine, based on a bias voltage applied to a source of the electromagnetic beam that impinged the position, the temperature of the heat treatment and the duration of time of the heat treatment. For example, at the operation 820, the product yield prediction module 214 can determine, using a machine learning technique, the prediction of the product yield. For example, the machine learning technique can include an unsupervised machine learning technique. For example, the machine learning technique can use a neural network.

Additionally, at an operation 824, for example, the synthesis controller module 218 can control, using the first data and the second data, the synthesis technique to produce the solid mixture.

Alternatively, at an operation 826, for example, the synthesis controller module 218 can communicate the first data and the second data to a controller configured to control the synthesis technique to produce the solid mixture.

Additionally, in FIG. 8A, in the method 800, at an operation 828, for example, the sample production module 220 can cause the system 200 to produce the initial sample of the solid mixture. For example, at the operation 828, the sample production module 220 can cause the system 200 to: (1) process, using a solvent, two or more solid state materials, (2) mix the two or more solid state materials, and (3) heat the two or more solid state materials. For example, the solvent can include ethanol (C₂H₆O). For example, the two or more solid state materials can include titanium dioxide (TiO₂) and lithium oxide (Li₂O). For example, at the operation 828, the sample production module 220 can mix, using a ball milling technique, the two or more solid state materials. For example, a specific end product produced by synthesizing the solid mixture can include lithium titanate (Li₂TiO₃).

Alternatively or additionally, for example, at the operation 828, the sample production module 220 can cause the system 200 to produce, using a robotic technique, the initial sample of the solid mixture. For example, the robotic technique can include moving the initial sample of the solid mixture to a stage of an electromagnetic beam management system.

Detailed embodiments are disclosed herein. However, one of skill in the art understands, in light of the description herein, that the disclosed embodiments are intended only as examples. Therefore, specific structural and functional details disclosed herein are not to be interpreted as limiting, but merely as a basis for the claims and as a representative basis for teaching one of skill in the art to variously employ the aspects herein in virtually any appropriately detailed structure. Furthermore, the terms and phrases used herein are not intended to be limiting but rather to provide an understandable description of possible implementations. Various embodiments are illustrated in FIGS. 1-7, 8A, and 8B, but the embodiments are not limited to the illustrated structure or application.

The flowchart and block diagrams in the figures illustrate the architecture, functionality, and operation of possible implementations of systems, methods, and computer program products according to various embodiments. In this regard, each block in flowcharts or block diagrams may represent a module, segment, or portion of code, which comprises one or more executable instructions for implementing the specified logical function(s). One of skill in the art understands, in light of the description herein, that, in some alternative implementations, the functions described in a block may occur out of the order depicted by the figures. For example, two blocks depicted in succession may, in fact, be executed substantially concurrently, or the blocks may be executed in the reverse order, depending upon the functionality involved.

The systems, components and/or processes described above can be realized in hardware or a combination of hardware and software and can be realized in a centralized fashion in one processing system or in a distributed fashion where different elements are spread across several interconnected processing systems. Any kind of processing system or another apparatus adapted for carrying out the methods described herein is suitable. A typical combination of hardware and software can be a processing system with computer-readable program code that, when loaded and executed, controls the processing system such that it carries out the methods described herein. The systems, components, and/or processes also can be embedded in a computer-readable storage, such as a computer program product or other data programs storage device, readable by a machine, tangibly embodying a program of instructions executable by the machine to perform methods and processes described herein. These elements also can be embedded in an application product that comprises all the features enabling the implementation of the methods described herein and that, when loaded in a processing system, is able to carry out these methods.

Furthermore, arrangements described herein may take the form of a computer program product embodied in one or more computer-readable media having computer-readable program code embodied, e.g., stored, thereon. Any combination of one or more computer-readable media may be utilized. The computer-readable medium may be a computer-readable signal medium or a computer-readable storage medium. As used herein, the phrase “computer-readable storage medium” means a non-transitory storage medium. A computer-readable storage medium may be, for example, but not limited to, an electronic, magnetic, optical, electromagnetic, infrared, or semiconductor system, apparatus, or device, or any suitable combination of the foregoing. More specific examples of the computer-readable storage medium would include, in a non-exhaustive list, the following: a portable computer diskette, a hard disk drive (HDD), a solid-state drive (SSD), a read-only memory (ROM), an erasable programmable read-only memory (EPROM or flash memory), a portable compact disc read-only memory (CD-ROM), a digital versatile disc (DVD), an optical storage device, a magnetic storage device, or any suitable combination of the foregoing. As used herein, a computer-readable storage medium may be any tangible medium that can contain or store a program for use by or in connection with an instruction execution system, apparatus, or device.

Generally, modules, as used herein, include routines, programs, objects, components, data structures, and so on that perform particular tasks or implement particular data types. In further aspects, a memory generally stores such modules. The memory associated with a module may be a buffer or may be cache embedded within a processor, a random-access memory (RAM), a ROM, a flash memory, or another suitable electronic storage medium. In still further aspects, a module as used herein, may be implemented as an application-specific integrated circuit (ASIC), a hardware component of a system on a chip (SoC), a programmable logic array (PLA), or another suitable hardware component (e.g., a central processing unit (CPU), a graphics processing unit (GPU), a field-programmable gate array (FPGA), or the like) that is embedded with a defined configuration set (e.g., instructions) for performing the disclosed functions.

Program code embodied on a computer-readable medium may be transmitted using any appropriate medium, including but not limited to wireless, wireline, optical fiber, cable, radio frequency (RF), etc., or any suitable combination of the foregoing. Computer program code for carrying out operations for aspects of the disclosed technologies may be written in any combination of one or more programming languages, including an object-oriented programming language such as Java™, Smalltalk, C++, or the like, and conventional procedural programming languages such as the “C” programming language or similar programming languages. The program code may execute entirely on a user's computer, partly on a user's computer, as a stand-alone software package, partly on a user's computer and partly on a remote computer, or entirely on a remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e., open language). The phrase “at least one of . . . or . . . ” as used herein refers to and encompasses any and all possible combinations of one or more of the associated listed items. For example, the phrase “at least one of A, B, or C” includes A only, B only, C only, or any combination thereof (e.g., AB, AC, BC, or ABC).

Aspects herein can be embodied in other forms without departing from the spirit or essential attributes thereof. Accordingly, reference should be made to the following claims, rather than to the foregoing specification, as indicating the scope hereof.

What is claimed is:

1. A system, comprising:
a processor; and
a memory storing:
a solid mixture sample assessment module including instructions that, when executed by the processor, cause the processor to determine a set of positions on interfaces between grains in an initial sample of a solid mixture, the set having maximal diversity of classifications of the interfaces in the initial sample;
an electromagnetic beam source control module including instructions that, when executed by the processor, cause electromagnetic beams to impinge the positions to produce an impinged sample, a characteristic of a first electromagnetic beam, which impinges a first position, being different from the characteristic of a second electromagnetic beam, which impinges a second position; and
a radiation-impinged sample assessment module including instructions that, when executed by the processor, cause the processor to analyze the positions on the impinged sample to determine information associated with synthesizing the solid mixture.

2. The system of claim 1, wherein a classification of an interface, of the classifications of the interfaces, comprises:
a first classification, the first classification being for a family of lattice planes of a first material at the interface; and
a second classification, the second classification being for a family of lattice planes of a second material at the interface.

3. The system of claim 1, wherein the characteristic comprises at least one of:
a bias voltage applied to a source of an electromagnetic beam of the electromagnetic beams, or
a size of a cross section of the electromagnetic beam.

4. The system of claim 1, wherein:
the solid mixture sample assessment module further includes instructions to produce an image of the initial sample, and
the instructions to determine the set of the positions include instructions to analyze the image of the initial sample to determine the set of the positions.

5. The system of claim 4, wherein:
the solid mixture sample assessment module further includes instructions to retain a calibration of the image of the initial sample, and
the instructions to cause the electromagnetic beams to impinge the positions include instructions to cause, using the calibration, the electromagnetic beams to impinge the positions.

6. The system of claim 4, wherein the instructions to produce the image of the initial sample include instructions to produce, using a scanning transmission electron microscopy technique, the image of the initial sample.

7. The system of claim 6, wherein a source of at least one of the electromagnetic beams comprises a scanning transmission electron microscope.

8. The system of claim 7, wherein the scanning transmission electron microscope is used to produce the image of the initial sample.

9. The system of claim 8, wherein:
at least one first bias voltage is applied to the scanning transmission electron microscope to produce the image of the initial sample, and
at least one second bias voltage is applied to the scanning transmission electron microscope to impinge the positions.

10. The system of claim 4, wherein the instructions to analyze the image of the initial sample include instructions to process, using an artificial intelligence technique, the image of the initial sample.

11. The system of claim 10, wherein the solid mixture sample assessment module further includes instructions to classify objects in the image of the initial sample, the objects including the interfaces.

12. The system of claim 11, wherein the objects further comprise grains of materials.

13. The system of claim 1, wherein:
the radiation-impinged sample assessment module further includes instructions to produce an image of the impinged sample, and
the instructions to analyze the positions on the impinged sample to determine the information associated with synthesizing the solid mixture include instructions to analyze the positions on the impinged sample, in the image of the impinged sample, to determine the information associated with synthesizing the solid mixture.

14. The system of claim 1, wherein the memory further stores:
a database management module including instructions that, when executed by the processor, cause the processor to store, in a database and for a position, of the positions, first data, the first data including:
the information associated with synthesizing the solid mixture,
a classification of an interface of the classifications of the interfaces,
the characteristic of an electromagnetic beam, of the electromagnetic beams, that impinged the position; and
a product yield prediction module including instructions that, when executed by the processor, cause the processor to:
obtain second data, the second data being associated with a production, using a synthesis technique, of the solid mixture; and
determine, using the first data and the second data, a prediction of a product yield of the production, using the synthesis technique, of the solid mixture.

15. The system of claim 14, wherein the memory further stores a synthesis controller module including instructions that, when executed by the processor, cause the processor to control, using the first data and the second data, the synthesis technique to produce the solid mixture.

16. The system of claim 1, wherein the memory further stores a sample production module including instructions that, when executed by the processor, cause the system to produce the initial sample of the solid mixture.

17. The system of claim 16, wherein the instructions to cause the system to produce the initial sample of the solid mixture include instructions to cause the system to produce, using a robotic technique, the initial sample of the solid mixture.

18. A method, comprising:
determining a set of positions on interfaces between grains in an initial sample of a solid mixture, the set having maximal diversity of classifications of the interfaces in the initial sample;
impinging, with electromagnetic beams, the positions to produce an impinged sample, a characteristic of a first electromagnetic beam, which impinges a first position, being different from the characteristic of a second electromagnetic beam, which impinges a second position; and
analyzing the positions on the impinged sample to determine information associated with synthesizing the solid mixture.

19. The method of claim 18, wherein the set of the positions on the interfaces has maximal diversity of the characteristics of the electromagnetic beams that impinge the positions.

20. A non-transitory computer-readable medium for determining information associated with synthesizing a solid mixture, the non-transitory computer-readable medium including instructions that, when executed by one or more processors, cause the one or more processors to:
determine a set of positions on interfaces between grains in an initial sample of the solid mixture, the set having maximal diversity of classifications of the interfaces in the initial sample;
cause electromagnetic beams to impinge the positions to produce an impinged sample, a characteristic of a first electromagnetic beam, which impinges a first position, being different from the characteristic of a second electromagnetic beam, which impinges a second position; and
analyze the positions on the impinged sample to determine the information associated with synthesizing the solid mixture.
